(19) United States
(12) Patent Application Publication
(10) Pub. No.: US 2021/0170083 A1
(43) Pub. Date: Jun. 10, 2021

(54) IMPLANTABLE MEDICAL DEVICE INCLUDING CABLE FASTENER

(71) Applicant: Medtronic, Inc., Minneapolis, MN (US)

(72) Inventors: Kevin Seifert, Forest Lake, MN (US); Yong K. Cho, Excelsior, MN (US); Michael D. Eggen, Chisago City, MN (US); David J. Peichel, Minneapolis, MN (US); Thomas W. Radtke, Ramsey, MN (US); Pankti Shah, Minneapolis, MN (US); Jason D. Hamack, Ramsey, MN (US)

(21) Appl. No.: 17/107,408

(22) Filed: Nov. 30, 2020

Related U.S. Application Data
(60) Provisional application No. 62/943,449, filed on Dec. 4, 2019.

(51) Int. Cl. A61M 1/12; H05K 5/02; H02J 50/00; H02J 50/10

(52) U.S. Cl. CPC A61M 1/127; H05K 5/0247; A61N 1/3975; H02J 50/10; A61M 1/122; H02J 50/005

(57) ABSTRACT

Various embodiments of an implantable medical device and a method of implanting such device are disclosed. The device includes a housing having a first major surface, a second major surface, a sidewall that extends between the first major surface and the second major surface, and a port disposed in the sidewall. The sidewall defines a perimeter of the housing. The device further includes an electronic component disposed within the housing, and a cable electrically connected to the electronic component disposed within the housing, where the cable extends through the port. A portion of the cable is adapted to be removably connected to the housing adjacent an outer surface of the sidewall by a fastener such that the portion of the cable extends along at least a portion of the perimeter of the housing when the portion of the cable is removably connected to the housing.

### CROSS-REFERENCE TO RELATED APPLICATIONS

This application claims the benefit of U.S. Provisional Application No. 62/943,449, filed Dec. 4, 2019, the disclosure of which is incorporated by reference herein in its entirety.

### TECHNICAL FIELD

This disclosure generally relates to various embodiments of an implantable medical device that includes a cable and a cable fastener.

### BACKGROUND

Various types of implantable medical devices can include a cable that extends between two or more components of the device to provide electrical energy and communication pathways between such components. These implantable medical devices can also include one or more components of a Transcutaneous Energy Transfer (TET) System. Such systems can be used to charge one or more batteries of an implantable medical device that is implanted internally within a human body. For example, a magnetic field generated by a transmitting coil outside the body can transmit power across a cutaneous (skin) barrier to a magnetic receiving coil implanted within the body. The receiving coil can transfer the received power to an implanted pump or other internal component or components and to one or more batteries implanted within the body to charge the battery.

Such systems can efficiently generate and wirelessly transmit a sufficient amount of energy to power one or more components of an implantable medical device while maintaining the system's efficiency and overall convenience of use.

### SUMMARY

The techniques of this disclosure generally relate to various embodiments of an implantable medical device that includes a cable and a cable fastener. The implantable medical device can be any suitable device that includes a cable that is electrically connected to one or more electronic components disposed within a housing of the device. The cable can extend through a port disposed in the housing. A portion of the cable can be adapted to be removably connected to the housing adjacent an outer surface of the housing by the fastener such that the portion of the cable extends along at least a portion of a perimeter of the housing when the cable is removably connected to the housing by the fastener.

In one example, aspects of this disclosure relate to an implantable medical device that includes a housing having a first major surface, a second major surface, a sidewall that extends between the first major surface and the second major surface, and a port disposed in the sidewall. The sidewall defines a perimeter of the housing. The device further includes an electronic component disposed within the housing, and a cable electrically connected to the electronic component disposed within the housing, where the cable extends through the port. A portion of the cable is adapted to be removably connected to the housing adjacent an outer surface of the sidewall by a fastener such that the portion of the cable extends along at least a portion of the perimeter of the housing when the portion of the cable is removably connected to the housing.

In another example, aspects of this disclosure relate to a wireless energy transfer system that includes an implantable medical device. The implantable medical device includes a housing having a first major surface, a second major surface, a sidewall that extends between the first major surface and the second major surface, and a port disposed in the sidewall. The sidewall defines a perimeter of the housing. The device further includes an electronic component disposed within the housing, and a cable electrically connected to the electronic component disposed within the housing, where the cable extends through the port. A portion of the cable is adapted to be removably connected to the housing adjacent an outer surface of the sidewall by a fastener such that the portion of the cable extends along at least a portion of the perimeter of the housing when the portion of the cable is removably connected to the housing.

In another example, aspects of this disclosure relate to a method that includes disposing an implantable medical device within a body of a patient, and determining a desired length of a cable of the implantable medical device. The cable is electrically connected to an electronic component disposed within a housing of the implantable medical device and extends through a port disposed in a sidewall of the device. The method further includes removably connecting a portion of the cable to the housing adjacent an outer surface of the sidewall utilizing a fastener such that the portion of the cable extends along at least a portion of a perimeter of the housing, where the perimeter of the housing is defined by the sidewall.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a perspective view of one embodiment of an implantable medical device.

FIG. 2 is a perspective view of a housing of the implantable medical device of FIG. 1.

FIG. 3 is a schematic cross-section view of the implantable medical device of FIG. 1.

FIG. 4 is a perspective view of another embodiment of an implantable medical device.

FIG. 5 is a schematic cross-section view of the implantable medical device of FIG. 4.

FIG. 6 is a schematic cross-section view of a portion of a resilient gasket of the implantable medical device of FIG. 4.

FIG. 7 is a perspective view of another embodiment of an implantable medical device.

FIG. 8 is a schematic cross-section view of the implantable medical device of FIG. 7.

FIG. 9 is a schematic top plan view of the implantable medical device of FIG. 7.

FIG. 10 is a schematic cross-section view of a portion of the implantable medical device of FIG. 7.

FIG. 11 is a schematic cross-section view of a portion of the implantable medical device of FIG. 7.

FIG. 12 is a schematic cross-section view of a portion of the implantable medical device of FIG. 7.

FIG. 13 is a schematic cross-section view of a portion of the implantable medical device of FIG. 7.

FIG. 14 is a schematic front view of external components of a wireless energy transfer system.

FIG. 15 is a schematic front view of an implantable medical device of the wireless energy transfer system of FIG. 14 disposed within a body of a patient.

FIG. 16 is a flowchart of one method of implanting the implantable medical device of FIG. 1.

### DETAILED DESCRIPTION

In general, the present disclosure provides various embodiments of an implantable medical device that includes a cable and a cable fastener. The implantable medical device can be any suitable device that includes a cable that is electrically connected to one or more electronic components disposed within a housing of the device. The cable can extend through a port disposed in the housing. A portion of the cable can be adapted to be removably connected to the housing adjacent an outer surface of the housing by the fastener such that the portion of the cable extends along at least a portion of a perimeter of the housing when the cable is removably connected to the housing by the fastener.

The various embodiments of implantable medical devices described herein can include any suitable medical device, e.g., a coil of an energy transfer system, a defibrillator, LVAD, neurostimulator, pacemaker, drug pump, etc. Further, the disclosed embodiments of implantable medical devices can be utilized with any suitable system or systems. For example, one or more embodiments of implantable medical devices can be utilized with a wireless energy transfer system, e.g., one or more of the systems described in U.S. Pat. No. 10,143,788 B2, entitled TRANSCUTANEOUS ENERGY TRANSFER SYSTEMS.

Some implantable medical devices such as charging coils for wireless energy transfer systems can include a cable that electrically connects two or more implanted components of the system. Varying lengths of the cable may, however, be required as locations of such components within the body can vary, and the physiology of each patient may be unique. For example, a cable may be a standard length; however, such length may be greater than a length needed to connect an implanted coil to an implanted electronic module. Excess length of the cable may undesirably move around within the patient or cause strain on the cable or the components connected to the cable.

One or more embodiments of an implantable medical devices described herein can allow a clinician to select a desired length of cable by connecting and securing at least a portion of the cable to a housing of the device with one or more fasteners. By securing a portion of the cable that is not required to connect two or more implanted components, strain relief may be provided to the cable and the connected components. Further, the one or more fasteners can hold the portion of cable in place adjacent a perimeter of the housing to prevent the cable from contacting a major surface of the housing, thereby minimizing heat transfer from the housing to the cable and vice versa, and also minimizing the additive effects of cable and housing heating, and increased contact pressure, on the surrounding tissue.

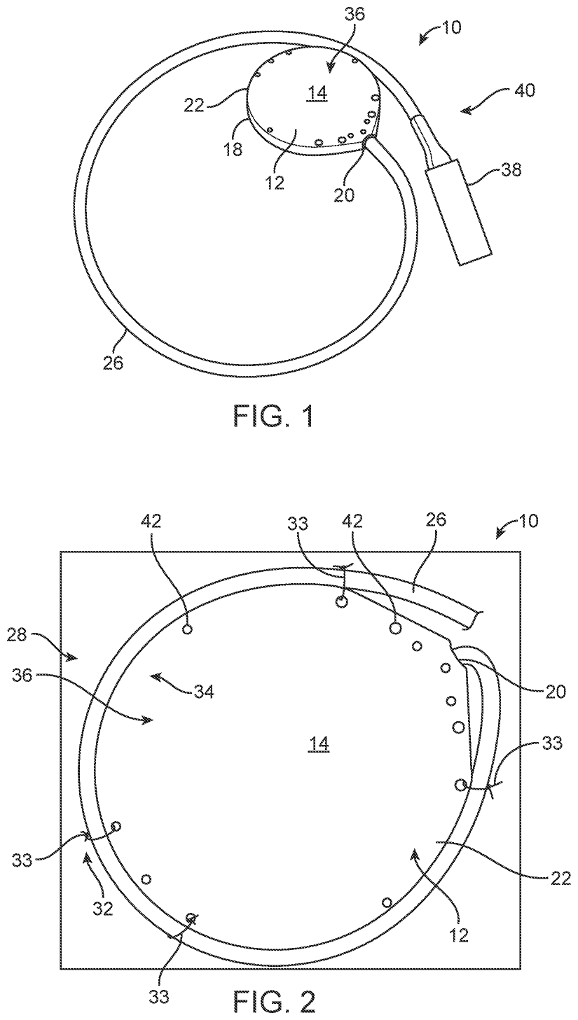

FIGS. 1-3 are various views of one embodiment of an implantable medical device 10. The device 10 includes a housing 12 having a first major surface 14, a second major surface 16, a sidewall 18 that extends between the first major surface and the second major surface, and a port 20 disposed in the sidewall. The sidewall 18 defines a perimeter 22 of the housing 12. The device 10 also includes an electronic component 24 disposed within the housing 12 and a cable 26 electrically connected to the electronic component inside the housing, where the cable extends through the port 20. As is further described herein, a portion 28 of the cable 26 is adapted to be removably connected to the housing 12 adjacent an outer surface 30 of the sidewall 18 by a fastener 32 such that the portion of the cable extends along at least a portion 34 of the perimeter 22 of the housing when the cable is removably connected to the housing.

The housing 12 can include any suitable material or materials, e.g., silicone, ceramic, polyurethane, or metal. Further, the housing 12 can take any suitable shape or shapes and have any suitable dimensions.

The first and second major surfaces 14,16 of the housing 12 can have any suitable dimensions and take any suitable shape or shapes. In one or more embodiments, at least one of the first major surface 14 or the second major surface 16 can take a planar shape. In one or more embodiments, at least one of the first major surface 14 or the second major surface 16 can take a curved shape.

The sidewall 18 of the housing 12 extends between the first major surface 14 and the second major surface 16. The sidewall 18 can have any suitable dimensions and take any suitable shape or shapes. For example, in the embodiment illustrated in FIGS. 1-3, the sidewall 18 takes a planar shape in a plane orthogonal to at least one of the first or second major surfaces 14,16. In one or more embodiments, the sidewall 18 can take a curved shape. In one or more embodiments, the sidewall 18 can have a concave (facing away from the housing 12) or convex (facing toward a center 36 of the housing) shape. Further, the sidewall 18 defines the perimeter 22 of the housing 12. In one or more embodiments, the first major surface 14 and the second major surface 16 may have curved edge portions that meet to define the sidewall 18. In such embodiments, the sidewall 18 is defined by an edge of the housing 12 formed by these curved edge portions of the first major surface 14 and the second major surface 16.

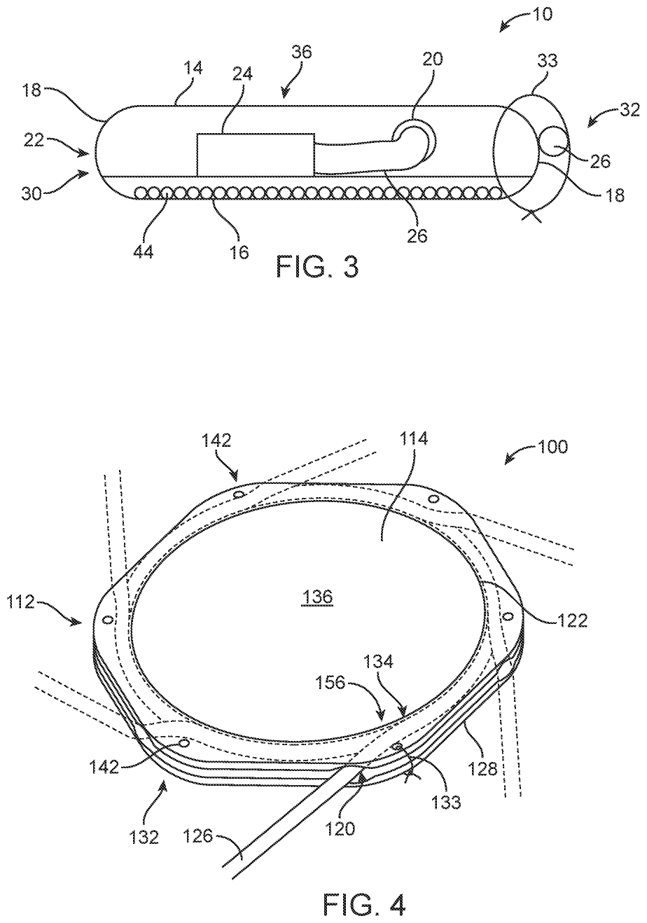

As shown in FIGS. 1-3, the port 20 is disposed in the sidewall 18 of the housing 12. The port 20 can take any suitable shape or shapes and have any suitable dimensions. In one or more embodiments, the port 20 is adapted to allow the cable 26 to extend therethrough such that the cable can be electrically connected to the electronic component 24 disposed within the housing 12 as is shown in FIG. 3. Although not shown, the port 20 can include a gasket or membrane that hermetically seals the port 20 to the cable 26.

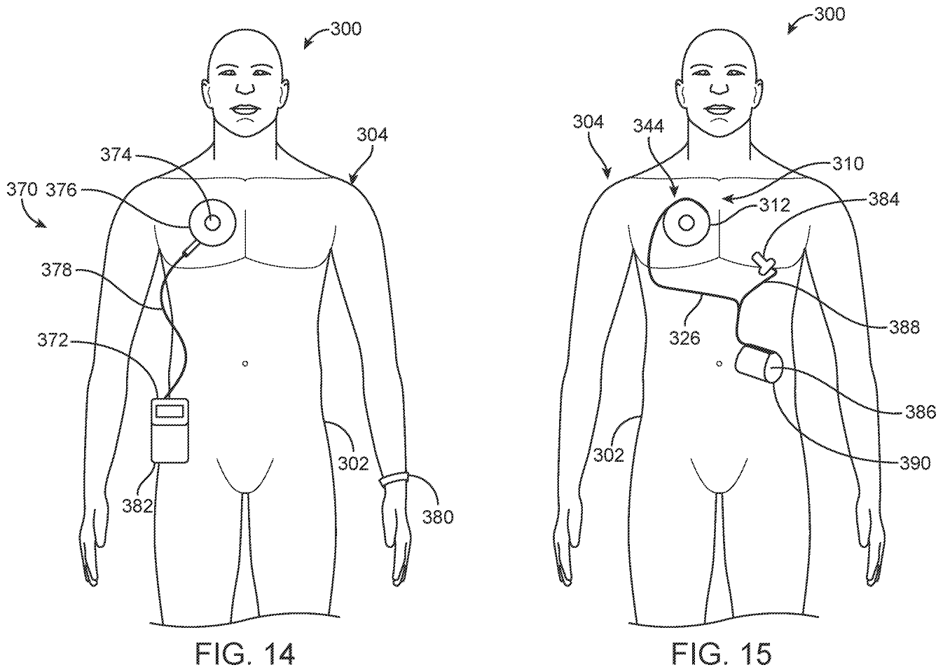

Disposed within the housing 12 is the electronic component 24. Although depicted as including one electronic component 24, the device 10 can include any suitable number of electronic components. Further, the electronic component 24 can include any suitable component or components, e.g., a capacitors, controllers, coils, tuning capacitors, pacemakers, defibrillators, etc. In one or more embodiments, the electronic component 24 can include a coil 44 that can be utilized with a wireless energy transfer system, e.g., wireless energy transfer system 300 of FIGS. 14-15. The electronic component 24 can be electrically connected to the cable 26 using any suitable technique or techniques.

Electrically connected to the electronic component 24 is the cable 26. The cable 26 can include any suitable material or materials, e.g., urethane, silicone, carbothane, MP35N, MP35N/silver core, etc. The cable 26 can include one or more conductors disposed within a protective sheath or covering. Such conductors can be electrically connected to the electronic device 24 using any suitable technique or techniques. The cable 26 can include any suitable number of conductors. Further, the cable 26 can have any suitable dimensions. The cable 26 can also have any suitable cross-sectional shapes, e.g., elliptical, rectangular, etc.

Although depicted as being connected to a single electronic component 24, the cable 26 can be connected to two or more electronic components disposed within the housing 12 of the device 10. Further, the cable 26 can include a connector 38 electrically connected to cable end 40. Such connector 38 can include any suitable connector that is adapted to connect the electronic component 24 disposed within the housing 12 to any suitable component or element of the device 10, e.g., a pump as is further described herein.

The cable 26 is electrically connected to the electronic component 24 disposed within the housing 12 and extends through the port 20 such that the cable exits the housing through the port. In one or more embodiments, the portion 28 of the cable 26 is adapted to be removably connected to the housing 12 adjacent the outer surface 30 of the sidewall 18. Any suitable technique or techniques can be utilized to connect the portion 28 of the cable 26 to the housing 12. In one or more embodiments, the portion 28 of the cable 26 can be connected to the housing 12 using a fastener 32 such that the portion of the cable extends along at least a portion 34 of the perimeter 22 of the housing 12 when the portion of the cable is removably connected to the housing.

The portion 28 of the cable 26 is removably connected to the housing 12 adjacent the outer surface 30 of the sidewall 18 by the fastener 32. As used herein, the term “adjacent the outer surface of the sidewall” means that a distance between an outer surface of the cable and the outer surface of the sidewall is no greater than 10 mm. In embodiments where a portion or portions of the cable 26 are wrapped around the housing 12 more than one time, the distance between the outermost portion of the cable and the sidewall 18 will be greater than when the cable is only wrapped once around the housing. In such embodiments, a distance between an outermost surface of the cable 26 and the outer surface 30 of the sidewall 18 is no greater than 20 mm. In one or more embodiments, the portion 28 of the cable 26 is disposed such that it is in contact with the sidewall 18. In one or more embodiments, the portion 34 of the perimeter 22 of the housing 12 along which the portion 28 of the cable 26 extends is at least 10% of the perimeter of the housing 12. Further, in one or more embodiments, this portion 34 is at least 25% of the perimeter 22 of the housing 12. In one or more embodiments, this portion 34 of the housing 12 can be no greater than 100%.

The fastener 32 can include any suitable fastening element or component that is adapted to removably connect the portion 28 of the cable 26 to the housing 12. Although not shown, the fastener 32 can be utilized to connect the housing 12 to tissue of the patient. In one or more embodiments, the fastener 32 can include one or more sutures 33 as shown in FIG. 2. The fastener 32 can include any suitable number of sutures 33. In one or more embodiments, the fastener 32 includes a plurality of sutures 33.

The sutures 33 can be connected to the housing 12 using any suitable technique or techniques. In one or more embodiments, the housing 12 can include one or more openings 42 disposed in at least one of the first major surface 14 or second major surface 16 of the housing. In one or more embodiments, the housing 12 can include a plurality of openings 42. In one or more embodiments, one or more of the openings 42 are disposed in each of the first and second major surfaces 14, 16 of the housing 12 such that the opening extends between the first major surface and the second major surface of the housing. Further, the openings 42 can be disposed in any suitable location in the housing 12. In one or more embodiments, the openings 42 can be disposed adjacent the perimeter 22 of the housing. As used herein, the term “adjacent the perimeter of the housing” means that an element or component is disposed within 10 mm of the perimeter of the housing.

To connect the portion 28 of the cable 26 to the housing 12, the fastener 32, e.g., suture 33, can be threaded through an opening 42 and wrapped around the portion of the cable. The ends of the suture 33 can be tied together such that the suture retains the cable 26. Any suitable technique or techniques can be utilized to thread and tie the suture 33. In one or more embodiments, two or more sutures 33 can be threaded through the same opening 42. In one or more embodiments, each suture 33 is connected to the housing 12 through an opening 42 of the plurality of openings. Further, additional sutures or other fasteners can be utilized to anchor the housing 12 to tissue of a patient using any suitable technique or techniques. In one or more embodiments, these sutures 33 can be threaded through one or more openings 42 in the housing 12 and through tissue.

The device 10 can also include the coil 44 disposed in any suitable location on or within the housing 12. The coil 44 can include any suitable material or materials and take any suitable shape or shapes. Further, the coil 44 can have any suitable dimensions and include any desired number of windings. In one or more embodiments, the coil 44 can be electrically connected to at least one of the electronic component 24 or the cable 26 using any suitable technique or techniques.

As mentioned herein, the fastener 32 can include any suitable element or component that is adapted to connect the portion 28 of the cable 26 to the housing 12. For example, FIGS. 4-6 are various views of another embodiment of an implantable medical device 100. All of the design considerations and possibilities regarding the medical device 10 of FIGS. 1-3 apply equally to the medical device 100 of FIGS. 4-6.

The implantable medical device 100 includes a housing 112 that has a first major surface 114 and a second major surface 116. The housing 112 also includes a sidewall 118 that extends between the first major surface 114 and the second major surface 116. The device 100 also includes an electronic component 124 disposed within the housing 112, and a cable 126 electrically connected to the electronic component, wherein the cable extends through a port 120. A portion 128 of the cable 126 is adapted to be removably connected to the housing 112 adjacent an outer surface 130 of the sidewall 118 by a fastener 132 such that the portion of the cable extends along at least a portion 134 of perimeter 122 of the housing when the portion of the cable is removably connected to the housing.

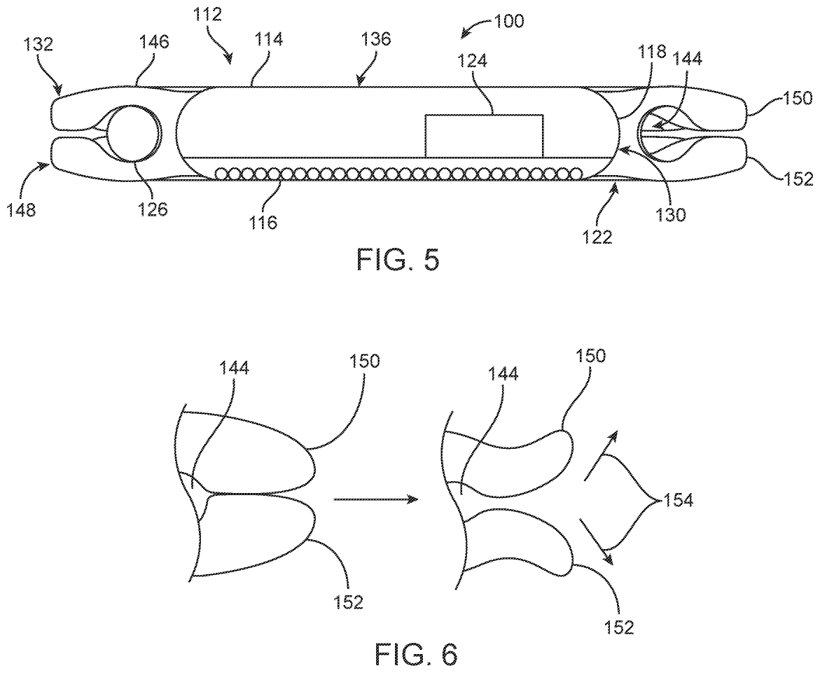

One difference between the implantable medical device 100 of FIGS. 4-6 and the implantable medical device 10 of FIGS. 1-3 is that the fastener 132 of the device 100 includes a slot 144 disposed adjacent the sidewall 118 of the housing 112. The slot 144 is adapted to retain the portion 128 of the cable 126 that is removably connected to the housing 112. Slot 144 can take any suitable shape or shapes and have any suitable dimensions. In one or more embodiments, the slot 144 can include a u-shape in a plane orthogonal to the first and second major surfaces 114,116 of the housing 112 as shown in FIG. 5. The slot 144 can retain the portion 128 of the cable 126 using any suitable technique or techniques. In one or more embodiments, the portion 128 of the cable 126 is friction-fit within the slot 144.

The slot 144 can be connected to the housing 112 using any suitable technique or techniques. In one or more embodiments, the slot 144 can be disposed in the sidewall 118 of the housing 112. Further, in one or more embodiments, a resilient gasket 146 can be disposed over the sidewall 118 of the housing 112, where the slot 144 is disposed in the resilient gasket as is shown in FIG. 5. The resilient gasket 146 can be attached or connected to the sidewall 118 of the housing 112 using any suitable technique or techniques, e.g., adhering, mechanically fastening, friction fitting, etc. In one or more embodiments, the resilient gasket 146 can be molded to the sidewall 118 of the housing 112 using any suitable technique or techniques. Further, the gasket 146 can be molded as part of the housing 112 such that the gasket and housing are integral. The gasket 146 can also be overmolded onto the housing 112 using any suitable technique or techniques. The gasket 146 can be continuous or segmented such that one or more portions of the sidewall 118 do not include a portion of the gasket.

In one or more embodiments, the sidewall 118 of the housing 112 can include one or more features that are adapted to assist in bonding or molding the resilient gasket 146 to the sidewall. Any suitable features can be disposed on the sidewall 118 to assist in such bonding or molding of the gasket 146.

The resilient gasket 146 can take any suitable shape or shapes and have any suitable dimensions. Further, the resilient gasket 146 can include any suitable material or materials, e.g., silicone, polyurethane, etc. In one or more embodiments, the resilient gasket 146 can include a reinforcing material disposed within a matrix, e.g., polyester mesh, polypropylene mesh, etc. Any suitable portion or portions of the gasket 146 can include a reinforcing material. For example, in one or more embodiments, one or both of a first tab 150 of the resilient gasket 146 and a second tab 152 of the gasket can include a reinforcing material. Further, the slot 144 of the gasket 146 can be reinforced with an eyelet that is inserted into or formed within the slot.

In one or more embodiments, the resilient gasket 146 includes an outer edge 148 that is adapted to deflect when engaging tissue of the patient to stabilize the device 100 within the tissue. For example, as illustrated in FIG. 5, the outer edge 148 of the resilient gasket 146 includes the first tab 150 and the second tab 152 disposed on each side of the slot 144. The first and second tabs 150, 152 are adapted to rotate away from the slot 144 (direction 154 in FIG. 6) when the outer edge 148 of the gasket 146 engages tissue of the patient.

The housing 112 can also include one or more openings 142 disposed in at least one of the first major surface 114 or second major surface 116 of the housing. Any suitable openings 142 can be disposed in the housing 112, e.g., openings 42 of device 10 of FIGS. 1-3. In one or more embodiments, one or more the openings 142 can be disposed in at least one of the first major surface 114 or second major surface 116 of the housing 112 adjacent the perimeter 122 of the housing. Further, in one or more embodiments, one or more the openings 142 can be disposed in the resilient gasket 146 adjacent the slot 144. As used herein, the phrase “adjacent the slot” means that the element or component is disposed closer to the slot 144 than to a center 136 of the housing 112 of the device 100 as shown in FIG. 5. In one or more embodiments, one or more openings 142 can be disposed through at least one of the first tab 150 or the second tab 152.

The fastener 132 can also include one or more sutures 133 that can be connected to the housing 112 through one or more openings 142 to further retain the cable 126. For example, a suture 133 can be threaded through an opening 142 at an exit 156 of the cable 126, i.e., where the cable exits the slot 144. Any suitable sutures 133 can be utilized, e.g., suture 33 of FIGS. 1-3. In one or more embodiments, the sutures 133 can provide strain relief to the cable 126 by securing it to the resilient gasket.

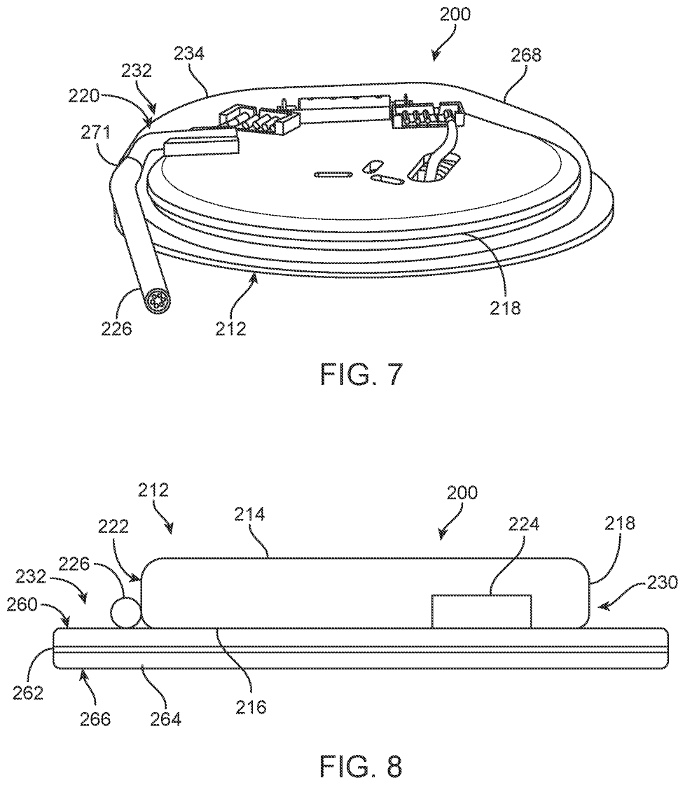

As mentioned herein, the various embodiments of fasteners are adapted to removably connect a portion of the cable to a housing of an implantable medical device. Further, one or more fasteners can be utilized to connect the housing of the device to tissue of a patient. For example, FIGS. 7-13 are various views of another embodiment of an implantable medical device 200. All of the design considerations and possibilities regarding the implantable medical device 10 of FIGS. 1-3 and the implantable medical device 100 of FIGS. 4-6 apply equally to the implantable medical device 200 of FIGS. 7-13.

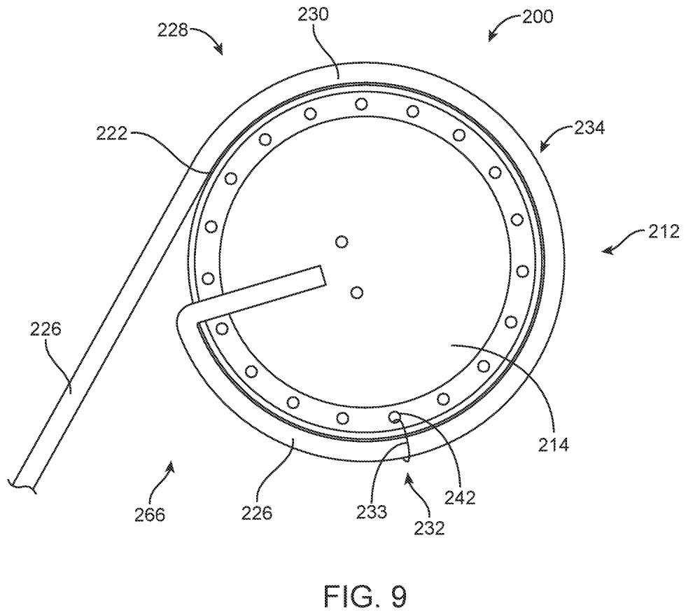

The device 200 includes a housing 212 having a first major surface 214, a second major surface 216, and a sidewall 218 that extends between the first major surface and the second major surface. The device 200 also includes an electronic component 224 disposed within the housing 212 and a cable 226 that is electrically connected to the electronic component and extends through a port 220 that is disposed in the sidewall 218 of the housing 212. A portion 228 of the cable 226 is adapted to be removably connected to the housing 212 adjacent an outer surface 230 of the sidewall 218 by a fastener 232 (FIG. 9) such that the portion of the cable extends along at least a portion 234 of a perimeter 222 of the housing when the portion of the cable is removably connected to the housing.

One difference between the implantable medical device 200 of FIGS. 7-9 and implantable medical devices 10 and 100 described herein is that a resilient substrate 260 is disposed on the second major surface 216 of the housing 212. A portion 266 of the resilient substrate 260 is adapted to extend over the portion 228 of the cable 226 that is connected to the housing 212 and be connected to the first major surface 214 of the housing, e.g., by a fastener 232. In one or more embodiments, the resilient substrate 260 is sized to wrap around a portion of the cable 226 to provide protection and strain relief to the cable.

The resilient substrate 260 can include any suitable material or materials, e.g., silicone, polyurethane, etc. In one or more embodiments, the substrate 260 can include a reinforcing layer 262 disposed on a major surface of the substrate or within the substrate as is shown in FIG. 8. In one or more embodiments, the reinforcing layer 262 can be disposed within a matrix 264 of the substrate 260. The reinforcing layer 262 can include any suitable material or materials, e.g., mesh (spunbonded, woven, etc.), woven or nonwoven layers, etc. Further, the matrix 264 can include any suitable material or materials, e.g., silicone, polyurethane, etc. The reinforcing layer 262 can be disposed within the matrix 264 using any suitable technique or techniques. In one or more embodiments, reinforcing layer 262 does not extend the entire length of the second major surface 216 but can be provided adjacent the perimeter 222 where reinforcement may be of benefit, such as a cord to improve the tear strength of the reinforcing layer.

In one or more embodiments, the substrate 260 can be connected to the second major surface 216 of the housing 212 using any suitable technique or techniques. In one or more embodiments, the substrate 260 can be, e.g., adhered, molded, or mechanically attached to the housing 212.

As mentioned herein, the portion 266 of the resilient substrate 260 can be folded or extended over the portion 228 of the cable 226 and connected to the first major surface 214 of the housing 212 by the fastener 232. The fastener 232 can include any suitable fastener fasteners, e.g., the same fasteners described herein regarding fastener 32 of FIG. 1-3. As shown in FIG. 9, the fastener 232 includes a suture 233 that can be extended through the folded portion 266 of the substrate 260 and an opening 242 that is disposed in the housing 212 as is further described herein. Any suitable number of fasteners 232 can be utilized to connect the portion 266 of the substrate 260 to the first major surface 214 of the housing 212. Although not shown, additional fasteners 232 can be utilized to attach additional portions of the cable 226 to the housing 212 using any suitable technique or techniques.

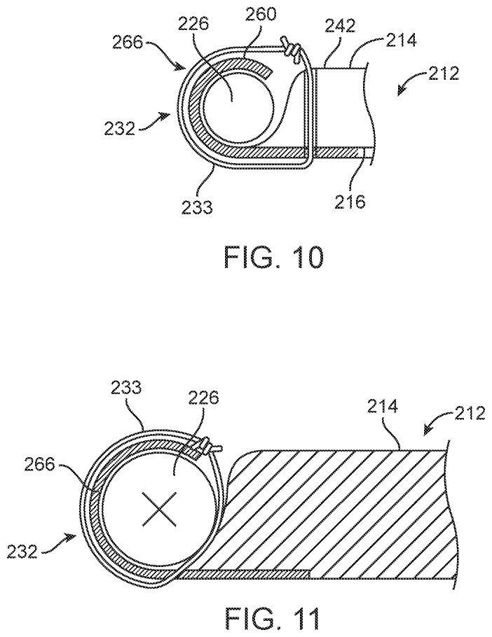

FIG. 10 illustrates one embodiment of the substrate 260 being utilized to retain the portion 228 of the cable 226. The portion 266 of the resilient substrate 260 is wrapped over the cable 226 and held in place by a suture 233 that is threaded through opening 242 and the substrate. In one or more embodiments, the suture 233 can be wrapped around the resilient substrate 260 and the cable 226 and threaded through the substrate 260 and not the housing 212 as shown in FIG. 11.

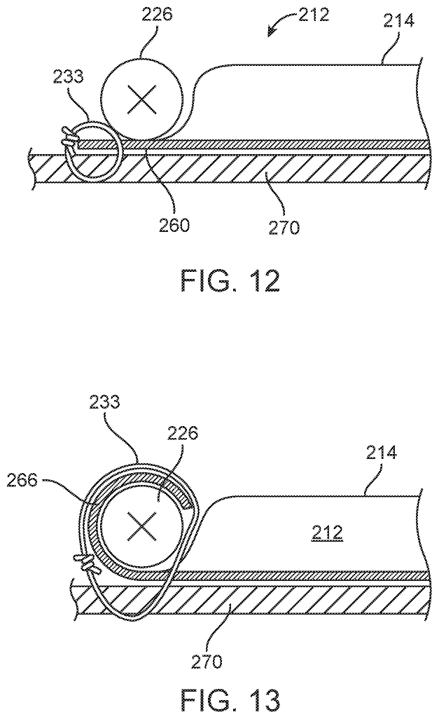

Further, additional sutures or other fasteners can be threaded through the resilient substrate 260 to anchor the housing 212 to tissue of a patient using any suitable technique or techniques. For example, as shown in FIG. 12, the suture 233 is threaded through the resilient substrate 260 and tissue 270 of the patient such that the housing 212 is connected to the tissue. In one or more embodiments, the suture 233 can be threaded through the resilient substrate 260 and tissue 270 with the substrate being wrapped around the cable 226 as shown in FIG. 13 to connect the cable 226 to the housing and the housing to the tissue 270.

Another difference between the implantable medical device 200 of FIGS. 7-13 and implantable medical devices 10 and 100 is that the housing 212 is encased in a resilient material 268. Any suitable resilient material or materials can be utilized, e.g., silicone, polyurethane, ceramic, metal, etc. Any suitable portion or portions of the housing 212 can be encased within the resilient material 268. In one or more embodiments, the entire housing 212 is encased within the resilient material 268, and an opening 271 can be formed in the resilient material such that the cable 226 extends through the resilient material.

The various embodiments of implantable medical devices described herein can be utilized with any suitable system. For example, FIGS. 14-15 are schematic views of one embodiment of a wireless energy transfer system 300. The system 300 includes an implantable medical device 310 and external components 370. In FIG. 14, the external components 370 of the system 300 are illustrated, and in FIG. 15, the implantable medical device 310 of the system is illustrated as being implanted within a body 302 of a patient 304. The external components 370 can include an external module 372 and a primary coil 374. In one or more embodiments, the primary coil 374 can be disposed in a separate housing 376 from the external module 372. The external module 372 can be located in any suitable location relative to the patient's body 302, e.g., around the patient's hip (e.g., in a pocket of the patient's clothing, mounted to a belt of the patient, etc.), and the primary coil 374 can be located in any suitable location relative to the patient's body 302, e.g., on the patient's chest and secured in place by a garment worn by the patient, such as a sling or vest. The external module 372 and primary coil 374 are further connected to each other by a wire 378. Also shown in FIG. 14 is a clinical monitor 380, which can be worn, e.g., on the patient's wrist. In other examples, the clinical monitor 380 can be located elsewhere, such as in the external module, or in the patient's smartphone, or not on the patient altogether.

In the embodiment illustrated in FIG. 14, an external battery and external electronics (not shown) can be disposed in a housing 382 of the external module 372. In one or more embodiments, the external battery may be disposed in a separate housing (e.g., separately mounted to the outside of the patient) and wired to the external module 372.

The implantable medical device 310 can include any suitable device described herein, e.g., device 10 of FIGS. 1-3. As illustrated in FIG. 15, the implantable medical device 310 can include a secondary coil 344 disposed within a housing 312, a pump 384, and an electronic module 386 electrically connected to the housing and the pump. In one or more embodiments, each of the housing 312, the pump 384, and the electronic module 386 can be disposed in a separate housing and dispersed throughout the patient's body 302 to accommodate the anatomy of the patient. For instance, in the embodiment illustrated in FIG. 15, the housing 312 is mounted in the patient's chest. In one or more embodiments, the housing 312 can be mounted to the patient's rib, back, abdomen, or muscle in any subcutaneous plane.

The housing 312 is electrically connected to the electronic module 386 by a first cable 326, and the pump 384 is electrically connected to the electronics module 386 by a second cable 388. The pump 384 can be connected, e.g., to a heart of the patient. Although not shown, the implantable medical device 310 can also include an implanted battery disposed in any suitable location within the patient's body 302. In one or more embodiments, the implanted battery is disposed within a housing 390 of the electronics module 386. In one or more embodiments, the implanted battery may be separately housed, and an additional wire may connect the electronics module 386 to the implanted battery.

The secondary coil 344 is disposed within the housing 312 of the implantable medical device 310 and is adapted to be electrically coupled to the primary coil 374. For example, the secondary coil 344 can be adapted to be inductively coupled to the primary coil 374. Positioning of the secondary coil 344 within the patient 304 can be done in such a manner that makes mounting the primary coil 374 in proximity to the secondary coil easy for the patient. For instance, the secondary coil 344 can be positioned close to the skin of the patient 304. Moreover, the secondary coil 344 can be positioned close to a relatively flat part of the patient's body 302 to make mounting the primary coil 374 easier. In the embodiment illustrated in FIG. 15, the secondary coil 344 disposed within the housing 312 is positioned close to the front of the patient's chest such that mounting the primary coil 374 to the patient's chest places the primary coil proximate the secondary coil. In those examples where the housing 312 is mounted to the patient's rib, back, or abdomen, the secondary coil 344 can similarly be located close to the patient's skin, such that the primary coil 374 can be mounted in close proximity.

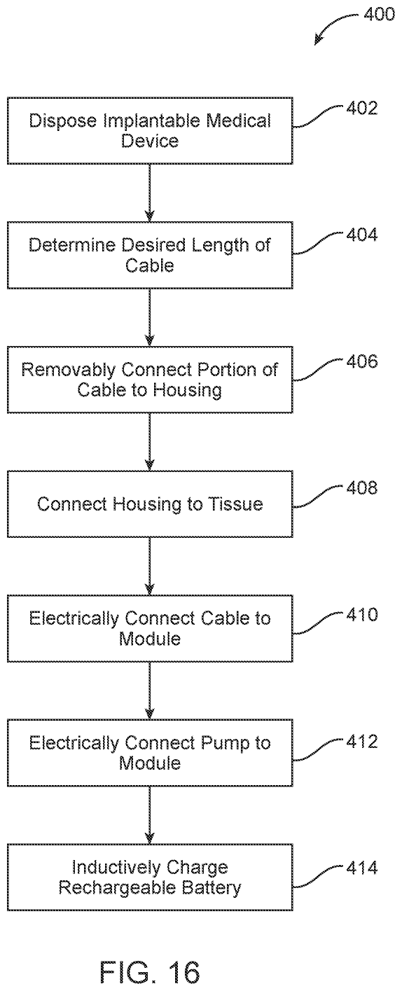

The various embodiments of implantable medical devices described herein can be implanted within a body of a patient using any suitable technique or techniques. For example, FIG. 16 is a flowchart of one embodiment 400 of a method 400 of implanting the implantable medical device 10 of FIGS. 1-3. Although described in regard to medical device 10, method 400 of FIG. 16 can be utilized with any implantable medical device. At 402, the implantable medical device 10 can be disposed within a body of a patient using any suitable technique or techniques. For example, an incision or incisions can be formed in any suitable portion of the body, and the implantable medical device 10 can be inserted into a cavity of the body through such incision. A desired length of the cable 26 of the implantable medical device 10 can be determined at 404. For example, a desired length of the cable 26 can include a distance from the implantable medical device 10 to an electronic module (e.g., electronic module 386 of FIG. 15) disposed within the body of the patient such that the cable 26 can electrically connect the electronic component 24 disposed within the housing 12 to the electronic module.

At 406, the portion 28 of the cable 26 is removably connected to the housing 12 adjacent the outer surface 30 of the sidewall 18 utilizing the fastener 32 such that the portion of the cable extends along at least a portion 34 of the perimeter 22 of the housing and such that the cable 26 has the desired length using any suitable technique or techniques. The housing 12 can be connected to tissue of the patient at 408 using any suitable technique or techniques.

In one or more embodiments, the cable 26 can optionally be electrically connected to an electronic module disposed within the body of the patient using any suitable technique or techniques at 410. In embodiments where the implantable medical device includes a pump (e.g., pump 384 of FIG. 15), such pump can be electrically connected to the electronic module with a second cable (e.g., second cable 388 of FIG. 15) disposed within the body of the patient at 412. Further, in embodiments where the implantable medical device 10 is a component of a wireless energy transfer system (e.g., system 300 of FIGS. 14-15), a battery disposed within the electronic module can be inductively recharged at 414 by disposing a primary coil adjacent a secondary coil disposed within the housing 12 of the implantable medical device using any suitable technique or techniques.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. An implantable medical device comprising:
a housing comprising a first major surface, a second major surface, a sidewall that extends between the first major surface and the second major surface, and a port disposed in the sidewall, wherein the sidewall defines a perimeter of the housing;
an electronic component disposed within the housing; and
a cable electrically connected to the electronic component disposed within the housing, wherein the cable extends through the port, and further wherein a portion of the cable is adapted to be removably connected to the housing adjacent an outer surface of the sidewall by a fastener such that the portion of the cable extends along at least a portion of the perimeter of the housing when the portion of the cable is removably connected to the housing.

2. The device of claim 1, wherein the fastener comprises a suture connected to the housing through an opening disposed in at least one of the first major surface or second major surface of the housing adjacent the perimeter of the housing.

3. The device of claim 1, wherein the fastener comprises a slot disposed adjacent the sidewall of the housing, wherein the slot is adapted to retain the portion of the cable that is removably connected to the housing.

4. The device of claim 3, further comprising a resilient gasket that is disposed over the sidewall of the housing, wherein the slot is disposed in the resilient gasket.

5. The device of claim 4, wherein the resilient gasket comprises an outer edge that is adapted to deflect when engaging tissue of a patient to stabilize the device within the tissue.

6. The device of claim 5, wherein the outer edge comprises first and second tabs disposed on each side of the slot, wherein the first and second tabs are adapted to rotate away from the slot when the outer edge of the gasket engages tissue of the patient.

7. The device of claim 1, wherein the portion of the cable extends along at least 10% of the perimeter of the housing.

8. The device of claim 1, wherein the fastener comprises a resilient mesh, wherein the housing is disposed within the resilient mesh.

9. The device of claim 1, further comprising a resilient substrate disposed on the second major surface of the housing, wherein a portion of the resilient substrate is adapted to extend over the portion of the cable and be connected to the first major surface of the housing by the fastener.

10. The device of claim 9, wherein the resilient substrate comprises a reinforcing layer disposed within a matrix.

11. The device of claim 10, wherein the reinforcing layer comprises a mesh.

12. The device of claim 9, wherein the fastener is connected to the housing and the resilient substrate.

13. The device of claim 1, wherein the housing is encased within a resilient material.

14. A wireless energy transfer system comprising an implantable medical device that comprises:
a housing comprising a first major surface, a second major surface, a sidewall that extends between the first major surface and the second major surface, and a port disposed in the sidewall, wherein the sidewall defines a perimeter of the housing;
an electronic component disposed within the housing; and
a cable electrically connected to the electronic component disposed within the housing, wherein the cable extends through the port, and further wherein a portion of the cable is adapted to be removably connected to the housing adjacent an outer surface of the sidewall by a fastener such that the portion of the cable extends along at least a portion of the perimeter of the housing when the portion of the cable is removably connected to the housing.

15. The system of claim 14, wherein the implantable medical device further comprises a coil disposed within the housing and electrically connected to the electronic component.

16. A method comprising:
disposing an implantable medical device within a body of a patient;
determining a desired length of a cable of the implantable medical device, wherein the cable is electrically connected to an electronic component disposed within a housing of the implantable medical device and extends through a port disposed in a sidewall of the device; and
removably connecting a portion of the cable to the housing adjacent an outer surface of the sidewall utilizing a fastener such that the portion of the cable extends along at least a portion of a perimeter of the housing, wherein the perimeter of the housing is defined by the sidewall.

17. The method of claim 16, further comprising electrically connecting the cable to an electronic module disposed within the body of the patient.

18. The method of claim 17, further comprising disposing a pump within the body of the patient such that the pump is connected to a heart of the patient.

19. The method of claim 18, further comprising electrically connecting the pump to the electronic module with a second cable disposed within the body of the patient.

20. The method of claim 16, further comprising connecting the housing to tissue of the patient.
